(19) United States
(12) Patent Application Publication
(10) Pub. No.: US 2015/0075521 A1
(43) Pub. Date: Mar. 19, 2015

(54) NEBULIZER AND MEDICAMENT STORAGE MODULE THEREOF

(71) Applicant: DELTA ELECTRONICS, INC., Taoyuan Hsien (TW)

(72) Inventors: Kuo-Liang Lee, Taoyuan Hsien (TW); Yu-Wei Lee, Taoyuan Hsien (TW)

(73) Assignee: DELTA ELECTRONICS, INC., Taoyuan Hsien (TW)

(21) Appl. No.: 14/144,181

(22) Filed: Dec. 30, 2013

(30) Foreign Application Priority Data
Sep. 13, 2013 (TW) 102133093

(51) Int. Cl. A61M 16/14; A61M 11/00

(52) U.S. Cl. CPC A61M 16/14; A61M 11/005; A61M 2205/123
USPC 128/200.16; 128/203.12

(57) ABSTRACT

A nebulizer includes a medicament storage module and a nebulizing unit. The medicament storage module includes an inclined bottom wall, a tubular sidewall, and a guiding part. The inclined bottom wall is inclined at an inclination angle. The tubular sidewall is connected with a periphery of the inclined bottom wall. A storage chamber is defined by the inclined bottom wall and the tubular sidewall collaboratively, and a liquid medicament is accommodated within the storage chamber. The tubular sidewall has an outlet, and the inclined bottom wall is extended downwardly to the outlet. The guiding part is arranged around a junction between the inclined bottom wall and the tubular sidewall. The nebulizing unit is disposed on the medicament storage module and aligned with the outlet. The liquid medicament outputted from the outlet is nebulized into a plurality of mist droplets by the nebulizing unit.

### FIELD OF THE INVENTION

The present invention relates to a nebulizer and a medicament storage module, and more particularly to a nebulizer and a medicament storage module that has reduced residual medicament liquid and is easily cleaned.

### BACKGROUND OF THE INVENTION

Recently, a nebulizer has been widely used for health care and beauty purposes. For example, the nebulizer may be used to administer medicament to treat or alleviate pain of the patient with a respiratory disease. In addition, the nebulizer may be used to administer medicament to be inhaled into the lungs of the patient in replace of oral administration. Consequently, the absorption efficiency and action efficiency of the medicament are enhanced and the occurrence of the side effect is reduced. Moreover, the nebulizer can be carried to immediately administer medicament and quickly generate the drug action to the patient with exacerbation of the obstructive respiratory tract. In other words, the use of the nebulizer can immediately cure or relieve the patient in the event of sudden onset.

Generally, the nebulizer may be used to nebulize or vaporize the liquid medicament into mist droplets. After these mist droplets are inhaled by the mouth of the patient, these mist droplets can reach bronchus and lungs of the patient for therapy and alleviation of the respiratory disease. According to the nebulizing mechanism, the nebulizers are classified into several types, e.g. a pneumatic jet nebulizer, an ultrasonic nebulizer and a mesh nebulizer. The nebulizing mechanism of the mesh nebulizer for nebulizing liquid medicament into mist droplets comprises a piezoelectric element, a nozzle plate and a corresponding micro pump.

FIG. 1 is a schematic cross-sectional view illustrating a medicament cartridge of a conventional mesh nebulizer. As shown in FIG. 1, the medicament cartridge 1 is substantially a rectangular hollow structure. The medicament cartridge 1 comprises a bottom wall 11, four lateral walls 12, an accommodation space 13, and an outlet 14. The bottom wall 11 is a rectangular plate. The four lateral walls 12 are connected with each other and perpendicular to each other. Moreover, the four lateral walls 12 are perpendicularly connected with the edges of the bottom wall 11. The accommodation space 13 is defined by the bottom wall 11 and the four lateral walls 12 collaboratively. The outlet 14 runs through one of the four lateral walls 12, and is located near the bottom wall 11. The liquid medicament within the accommodation space 13 can be outputted or supplied through the outlet 14.

In the conventional medicament cartridge 1, the bottom wall 11 is in parallel with the horizontal plane, and the four lateral walls 12 are perpendicularly connected with the edges of the bottom wall 11. Consequently, a plurality of inside corners are formed at the bottom of the accommodation space 13. Due to the inside corners, a portion of the liquid medicament is usually retained at the bottom of the accommodation space 13 after the nebulizing operation of the nebulizer is completed. In other words, the conventional medicament cartridge 1 may result in the problems of wasting medicament or supplying insufficient amount of medicament. Moreover, since the medicament contained in the accommodation space 13 of the medicament cartridge 1 is viscous and the inside corners at the bottom of the accommodation space 13 are dead zones of liquid flow, the medicament is readily retained at the regions of the bottom wall 11 far from the outlet 14 and at the inside corners. Moreover, it is difficult to clean the conventional medicament cartridge 1. The subsequent use of the medicament cartridge 1 may contaminate the medicament or cause a bacteria problem.

Therefore, there is a need of providing a nebulizer and a medicament storage module in order to eliminate the above drawbacks.

### SUMMARY OF THE INVENTION

The present invention provides a nebulizer and a medicament storage module for solving the problems of wasting medicament or supplying insufficient medicament.

The present invention provides a nebulizer and a medicament storage module that are easily cleaned. In the subsequent use of the nebulizer, the problem of contaminating the medicament or causing bacteria growth will be avoided.

In accordance with an aspect of the present invention, there is provided a nebulizer. The nebulizer includes a medicament storage module and a nebulizing unit. The medicament storage module includes an inclined bottom wall, a tubular sidewall, and a guiding part. The inclined bottom wall is inclined at an inclination angle with respect to a horizontal plane. The tubular sidewall is connected with a periphery of the inclined bottom wall. A storage chamber with a top entrance is defined by the inclined bottom wall and the tubular sidewall collaboratively, and a liquid medicament is accommodated within the storage chamber. The tubular sidewall has an outlet, and the inclined bottom wall is extended downwardly to the outlet. The guiding part is arranged around a junction between the inclined bottom wall and the tubular sidewall. The nebulizing unit is disposed on the medicament storage module and aligned with the outlet. The liquid medicament outputted from the outlet is nebulized into a plurality of mist droplets by the nebulizing unit.

In accordance with another aspect of the present invention, there is provided a medicament storage module. The medicament storage module includes an inclined bottom wall, a tubular sidewall, and a guiding part. The inclined bottom wall is inclined at an inclination angle with respect to a horizontal plane. The tubular sidewall is connected with a periphery of the inclined bottom wall. A storage chamber with a top entrance is defined by the inclined bottom wall and the tubular sidewall collaboratively, and a liquid medicament is accommodated within the storage chamber. The tubular sidewall has an outlet, and the inclined bottom wall is extended downwardly to the outlet. The guiding part is arranged around a junction between the inclined bottom wall and the tubular sidewall.

In accordance with a further aspect of the present invention, there is provided a medicament storage module. The medicament storage module includes an inclined bottom wall, a tubular sidewall, and a C-shaped curvy structure. The inclined bottom wall is inclined at an inclination angle with respect to a horizontal plane. The C-shaped curvy structure is arranged around a junction between the inclined bottom wall and the tubular sidewall. A storage chamber with a top entrance and an outlet is defined by the inclined bottom wall, the tubular sidewall and the C-shaped curvy structure collaboratively. A liquid medicament is accommodated within the storage chamber. The inclined bottom wall is extended downwardly to the outlet.

The above contents of the present invention will become more readily apparent to those ordinarily skilled in the art after reviewing the following detailed description and accompanying drawings, in which:

### BRIEF DESCRIPTION OF THE DRAWINGS

FIG. 1 is a schematic cross-sectional view illustrating a medicament cartridge of a conventional mesh nebulizer;

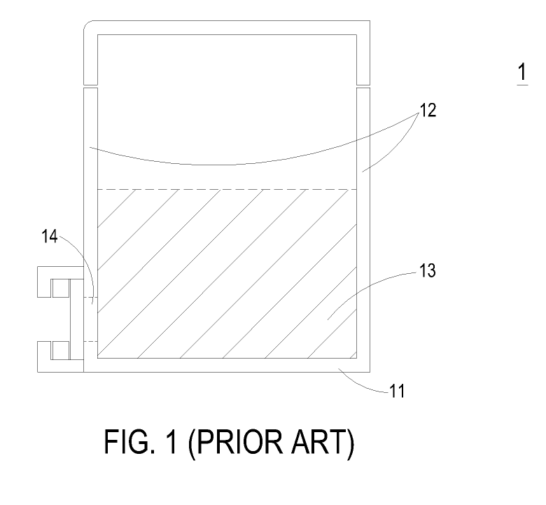

FIG. 2A is a schematic side view illustrating a nebulizer according to an embodiment of the present invention;

FIG. 2B is a schematic cross-sectional view illustrating the nebulizer of FIG. 2A and taken along the line AA;

FIG. 3A is a schematic perspective view illustrating the medicament storage module of the nebulizer of FIGS. 2A and 2B;

FIG. 3B is a schematic perspective view illustrating the medicament storage module of FIG. 3A and taken along another viewpoint;

FIG. 3C is a schematic top view illustrating the medicament storage module of FIG. 3A;

FIG. 3D is a schematic cross-sectional view illustrating the medicament storage module of FIG. 3A and taken along the line BB;

FIG. 4A is a schematic perspective view illustrating a variant example of the medicament storage module of the nebulizer of the present invention, wherein the upper cover is not shown;

FIG. 4B is a schematic cross-sectional view illustrating the medicament storage module of FIG. 4A and taken along the line DD; and

FIG. 5 is a plot illustrating the relationship between the residual medicament percentage and the inclination angle of the inclined bottom wall of the medicament storage module of FIGS. 2A and 2B.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

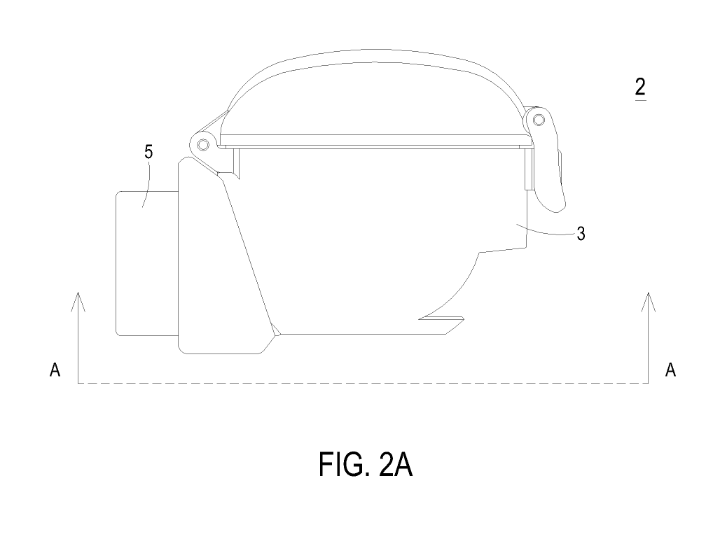

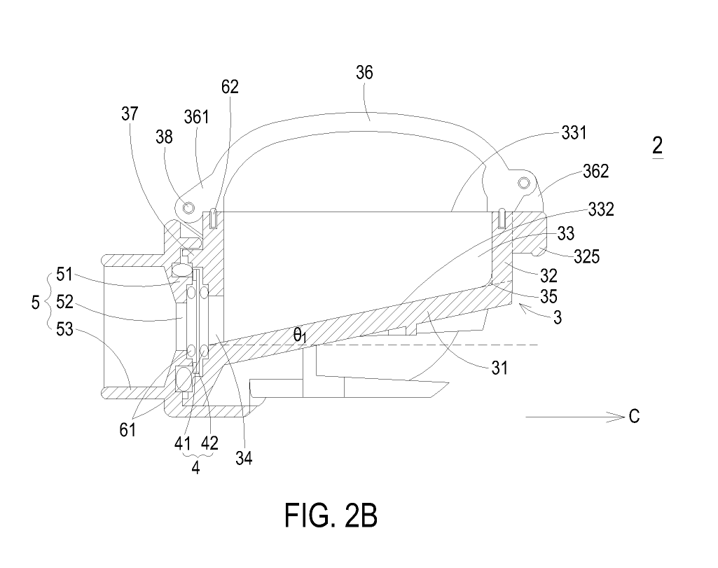

FIG. 2A is a schematic side view illustrating a nebulizer according to an embodiment of the present invention. FIG. 2B is a schematic cross-sectional view illustrating the nebulizer of FIG. 2A and taken along the line AA. An example of the nebulizer 2 includes but is not limited to a mesh nebulizer. The nebulizer 2 is used for nebulizing liquid medicament into a plurality of tiny mist droplets. For example, the mist droplets have a diameter in the range between 1 micrometer and 5 micrometers. After these mist droplets are inhaled by the mouth of the patient, these mist droplets can reach bronchus and lungs of the patient for therapy and alleviation of the respiratory disease.

In this embodiment, the nebulizer 2 comprises a medicament storage module 3, a nebulizing unit 4, and a conduit 5. The medicament storage module 3 is used for containing and supplying liquid medicament. The nebulizing unit 4 comprises a piezoelectric element 41 and a nozzle plate 42. The nebulizing unit 4 is disposed on the medicament storage module 3. By the nebulizing unit 4, the liquid medicament from the medicament storage module 3 is nebulized into the tiny mist droplets to be ejected. The conduit 5 is detachably connected with the medicament storage module 3, and aligned with the nebulizing unit 4. By the conduit 5, the tiny mist droplets ejected from the nebulizing unit 4 is guided to the mouth of the patient.

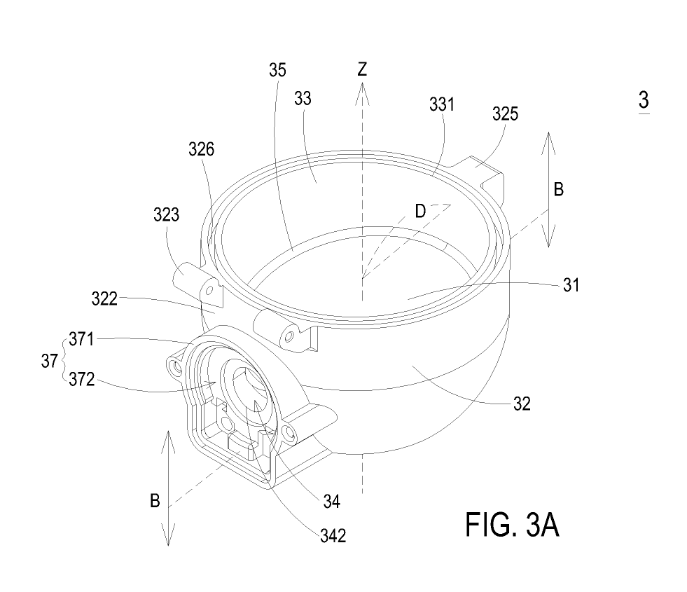

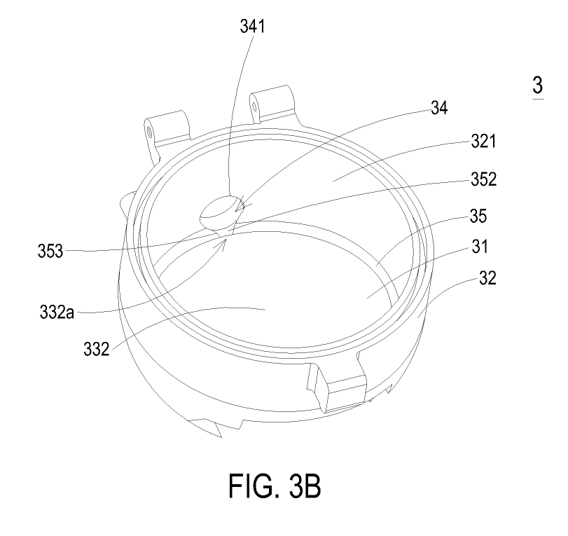

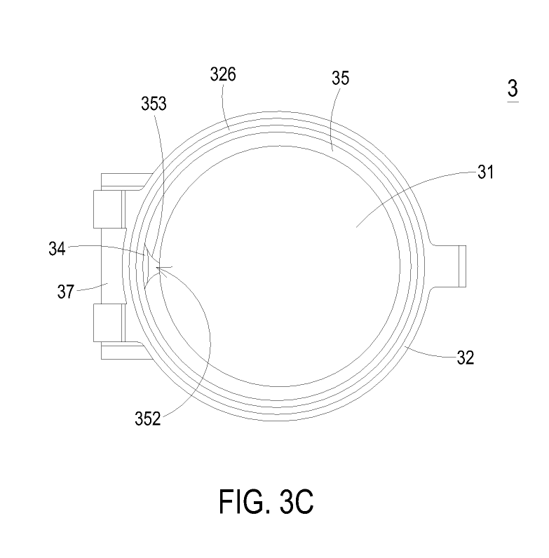

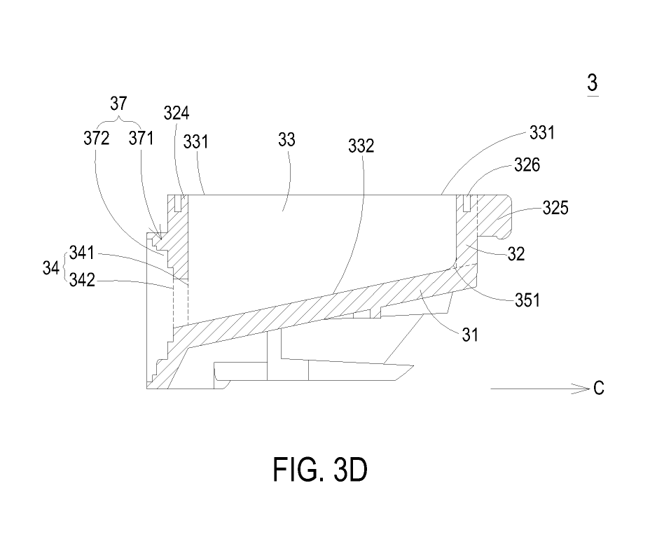

FIG. 3A is a schematic perspective view illustrating the medicament storage module of the nebulizer of FIGS. 2A and 2B. FIG. 3B is a schematic perspective view illustrating the medicament storage module of FIG. 3A and taken along another viewpoint. FIG. 3C is a schematic top view illustrating the medicament storage module of FIG. 3A. FIG. 3D is a schematic cross-sectional view illustrating the medicament storage module of FIG. 3A and taken along the line BB. Please refer to FIGS. 2A, 2B, 3A, 3B, 3C and 3D. The medicament storage module 3 has a cup-shaped structure. In addition, the medicament storage module 3 comprises an inclined bottom wall 31, a tubular sidewall 32, a storage chamber 33, an outlet 34, and a guiding part 35. The inclined bottom wall 31 is inclined at an inclination angle θ1 with respect to a horizontal plane C. The inclination angle θ1 is in the range between 3 and 30 degrees, and preferably in the range between 5 and 15 degrees. The tubular sidewall 32 is connected with a periphery of the inclined bottom wall 31. Moreover, the storage chamber 33 has a top entrance 331 at a top side thereof. The storage chamber 33 is defined by the inclined bottom wall 31 and the tubular sidewall 32. The storage chamber 33 is used for accommodating or storing the liquid medicament. In an embodiment, the tubular sidewall 32 and the inclined bottom wall 31 are integrally formed with each other, and the tubular sidewall 32 is protruded upwardly from the periphery of the inclined bottom wall 31. Preferably, the tubular sidewall 32 is a circular sidewall. That is, the tubular sidewall 32 or the storage chamber 33 has a center axis Z. The center axis Z is perpendicular to the horizontal plane C. Moreover, there is an equidistance D between the center axis Z and the tubular sidewall 32. In this embodiment, the thickness and the diameter of the inclined bottom wall 31 and the thickness and the height of the tubular sidewall 32 may be varied according to the practical requirements.

The guiding part 35 of the medicament storage module 3 is located at a bottom surface 332 of the storage chamber 33, and arranged around the junction between the inclined bottom wall 31 and the tubular sidewall 32. In other words, the guiding part 35 is arranged around the periphery of the bottom surface 332 of the storage chamber 33. By means of the guiding part 35, no inside corners are formed within the storage chamber 33 of the medicament storage module 3. Consequently, the possibility of retaining the liquid medicament in the storage chamber 33 can be reduced, and the storage chamber 33 can be easily cleaned. In this embodiment, the guiding part 35 is a C-shaped curvy structure, but is not limited thereto. Preferably, the C-shaped curvy structure 35 has a concave curvy surface 351. Moreover, the radian of the curvy surface 351 of the C-shaped curvy structure 35 is not restricted, and may be varied according to the practical requirements. In some embodiments, the inclined bottom wall 31, the tubular sidewall 32 and the guiding part 35 are integrally formed with each other.

The outlet 34 is formed in the tubular sidewall 32 of the medicament storage module 3. That is, the outlet 34 runs through the tubular sidewall 32. The liquid medicament within the storage chamber 33 can be outputted or supplied through the outlet 34. The outlet 34 has a first opening 341 and a second opening 342. The first opening 341 is open to an inner surface 321 of the tubular sidewall 32 and aligned with a notch 352 of the guiding part 35. The second opening 342 is open to an outer surface 322 of the tubular sidewall 32. In this embodiment, the inclined bottom wall 31 is extended downwardly to the outlet 34 and aligned with the nebulizing unit 4. The first opening 341 of the outlet 34 is close to a lowest position 332a of the bottom surface 332 of the storage chamber 33 (see FIG. 3B). The distance between the lowest position 332a of the bottom surface 332 of the storage chamber 33 and the horizontal plane C is the shortest among all distances between the bottom surface 332 of the storage chamber 33 and the horizontal plane C. In an embodiment, the both sides of the notch 352 of the guiding part 35 have beveled edges 353 for facilitating guiding the liquid medicament to the outlet 34.

The medicament storage module 3 further comprises a coupling part 37. The coupling part 37 is connected with the outer surface 322 of the tubular sidewall 32 and aligned with the second opening 342 of the outlet 34. After the nebulizing unit 4 is installed in the coupling part 37, the nozzle plate 42 of the nebulizing unit 4 is aligned with the second opening 342 of the outlet 34. In an embodiment, the coupling part 37 and the tubular sidewall 32 are integrally formed with each other, and the coupling part 37 is extended externally from the outer surface 322 of the tubular sidewall 32 and aligned with the second opening 342 of the outlet 34. The coupling part 37 comprises an extension wall 371 and a first recess 372. The first recess 372 is defined by the extension wall 371 and the tubular sidewall 32 collaboratively. Moreover, the lateral surface of the first recess 372 has a multi-stepped structure mating with the piezoelectric element 41 and the nozzle plate 4 of the nebulizing unit 4, so that the nozzle plate 42 is aligned with the second opening 342 of the outlet 34.

The conduit 5 is detachably connected with the coupling part 37. In this embodiment, the conduit 5 comprises a coupling part 51, an entrance 52, and a tube part 53. The coupling part 51 of the conduit 5 is coupled with the extension wall 371 of the coupling part 37, and fixed on the coupling part 37. The entrance 52 of the conduit 5 is aligned with the nozzle plate 42 of the nebulizing unit 4. After the mist droplets are ejected from the nebulizing unit 4, the mist droplets are guided by the tube part 53 of the conduit 5. In some embodiments, one or more waterproof gaskets 61 (e.g. ring-shaped rubbery gaskets) are arranged between the nebulizing unit 4 and the outer surface 322 of the tubular sidewall 32 and between the nebulizing unit 4 and the coupling part 51 of the conduit 5 in order to prevent leakage of the liquid medicament.

The medicament storage module 3 further comprises an upper cover 36. The upper cover 36 is pivotally coupled to the tubular sidewall 32 for sheltering the top entrance 331 of the storage chamber 33. The tubular sidewall 32 further comprises at least one first shaft sleeve 323, and the upper cover 36 further comprises at least one second shaft sleeve 361. The medicament storage module 3 further comprises at least one shaft lever 38. After the shaft lever 38 is penetrated through the first shaft sleeve 323 and the second shaft sleeve 361, the upper cover 36 is pivotally coupled to the tubular sidewall 32. In a preferred embodiment, the first shaft sleeve 323 is integrally formed with the tubular sidewall 32, and located near a top edge 324 or the entrance 321 of the tubular sidewall 32, and located near the coupling part 37. Moreover, the tubular sidewall 32 further comprises at least one first engaging element 325, and the upper cover 36 comprises at least one second engaging element 362 corresponding to the at least one first engaging element 325. After the upper cover 36 is rotated to the position over the top entrance 331 of the storage chamber 33, the first engaging element 325 of the tubular sidewall 32 and the second engaging element 362 of the upper cover 36 are engaged with each other. Consequently, the top entrance 331 of the storage chamber 33 is tightly sealed by the upper cover 36. In an embodiment, the first engaging element 325 is located near the top edge 324 or the entrance 321 of the tubular sidewall 32, and is opposed to the first shaft sleeve 323. Moreover, a second recess 326 is formed in the top edge 324 of the tubular sidewall 32. A waterproof gasket 62 (e.g. a ring-shaped rubbery gasket) is accommodated within the second recess 326 in order to prevent leakage of the liquid medicament. In some other embodiments, the upper cover 36 further comprises one or more ventilation holes (not shown), but is not limited thereto.

The nebulizing unit 4 comprises the piezoelectric element 41 and the nozzle plate 42. The piezoelectric element 41 is a vibrating element for driving vibration of the nozzle plate 42. The nozzle plate 42 comprises a plurality of nozzles (not shown). As the nozzle plate 42 is vibrated by the piezoelectric element 41, the liquid medicament is nebulized into the tiny mist droplets to be ejected through the nozzles. The piezoelectric element 41 and the nozzle plate 4 of the nebulizing unit 4 are disposed within the first recess 372 of the coupling part 37 of the medicament storage module 3. In addition, the nozzle plate 42 is aligned with the second opening 342 of the outlet 34. Consequently, the liquid medicament outputted from the outlet 34 can be nebulized into the tiny mist droplets by the nebulizing unit 4. In an embodiment, the nozzle plate 42 has a circular profile. In particular, the nozzles of the nozzle plate 42 (i.e. the effective action region) are directly aligned with the second opening 342 of the outlet 34. Consequently, the nebulizing efficiency of the nebulizer 2 is enhanced.

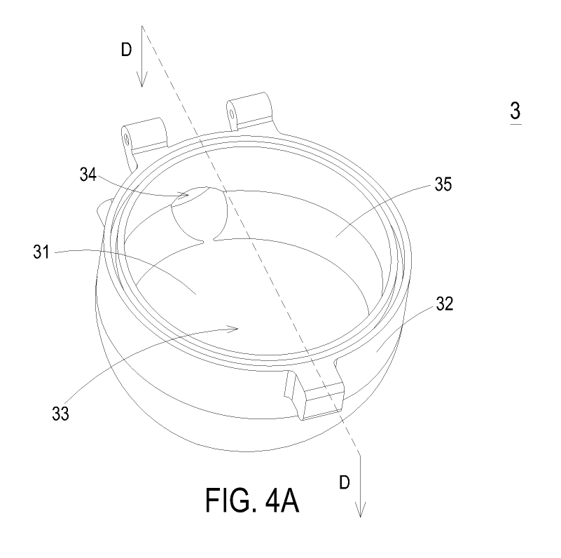

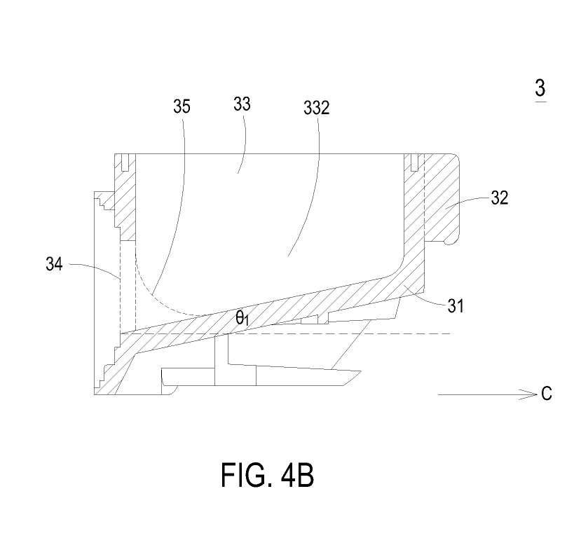

FIG. 4A is a schematic perspective view illustrating a variant example of the medicament storage module of the nebulizer of the present invention, wherein the upper cover is not shown. FIG. 4B is a schematic cross-sectional view illustrating the medicament storage module of FIG. 4A and taken along the line DD. As shown in FIGS. 4A and 4B, the medicament storage module 3 has a cup-shaped structure. In addition, the medicament storage module 3 comprises an inclined bottom wall 31, a tubular sidewall 32, a storage chamber 33, an outlet 34, and a C-shaped curvy structure 35. The inclined bottom wall 31 is inclined at an inclination angle θ1 with respect to a horizontal plane C. The inclination angle θ1 is in the range between 3 and 30 degrees, and preferably in the range between 5 and 15 degrees. The storage chamber 33 with a top entrance 331 and the outlet 34 is defined by the inclined bottom wall 31, the tubular sidewall 32 and the C-shaped curvy structure 33 collaboratively. The inclined bottom wall 31 is extended downwardly to the outlet 34. The C-shaped curvy structure 35 is connected with the inclined bottom wall 31 and tubular sidewall 32, and arranged around the periphery of the bottom surface 332 of the storage chamber 33. By means of the guiding part 35, no inside corners are formed within the storage chamber 33 of the medicament storage module 3. Consequently, the possibility of retaining the liquid medicament in the storage chamber 33 can be reduced, and the storage chamber 33 can be easily cleaned. The structures and functions of the inclined bottom wall 31, the tubular sidewall 32, the storage chamber 33, the outlet 34 and the C-shaped curvy structure 35 of this embodiment are similar to those illustrated in FIGS. 3A, 3B, 3C and 3D, and are not redundantly described herein.

In some embodiments, the nebulizer 2 further comprises a handle (not shown), and the handle is detachably connected with the medicament storage module 3. Moreover, a power source (not shown) may be disposed within the handle for providing electric power to associated components of the nebulizer 2. Moreover, a control panel (not shown) may be disposed within the handle for controlling operations of the nebulizer 2.

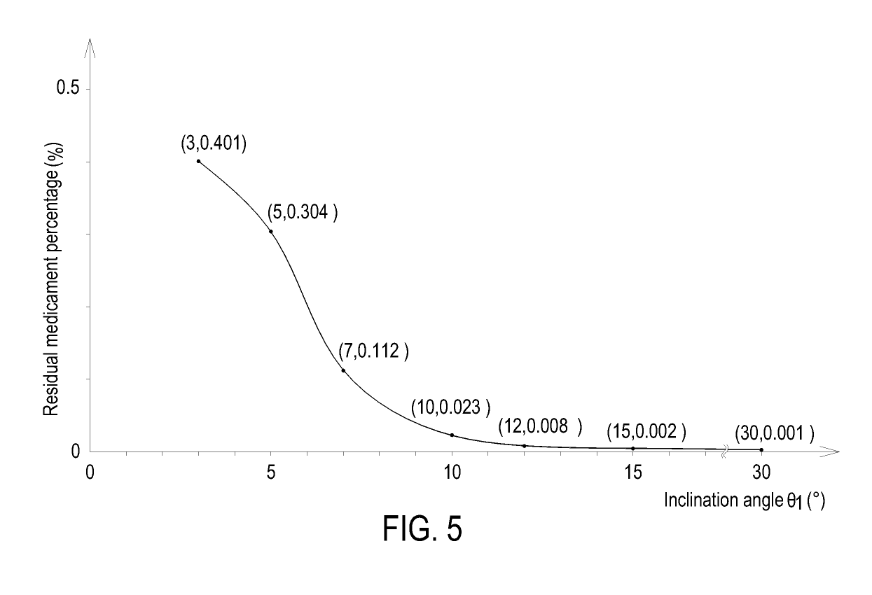

FIG. 5 is a plot illustrating the relationship between the residual medicament percentage and the inclination angle of the inclined bottom wall of the medicament storage module of FIGS. 2A and 2B. In case that the inclination angle θ1 of the inclined bottom wall 31 of the medicament storage module 3 is 3, 5, 7, 10, 12, 15 and 30 degrees, the residual medicament percentage is 0.401%, 0.304%, 0.112%, 0.023%, 0.008%, 0.002% and 0.001%, respectively. That is, if the inclination angle θ1 of the inclined bottom wall 31 in the range between 3 and 30 degrees, the residual medicament percentage of the nebulizer 2 can be effectively reduced. Especially, if the inclination angle 81 of the inclined bottom wall 31 in the range between 5 and 15 degrees, the overall height and size of the nebulizer 2 and the storage chamber 33 of the medicament storage module 3 can be reduced while complying with the required medicament capacity (e.g. 4˜5 ml). Consequently, the nebulizer 2 can be carried by the user more easily.

From the above descriptions, the present invention provides a nebulizer and a medicament storage module. By means of a tubular sidewall and an inclined bottom wall with an inclination angle, the viscous liquid medicament can be easily guided to the outlet and outputted. By means of a guiding part (e.g. a C-shaped curvy structure) of the medicament storage module, no inside corners are formed within the storage chamber of the medicament storage module. Consequently, there are no dead zones in the storage chamber, and the liquid medicament can be naturally guided to the outlet by the guiding part. After the nebulizing operation of the nebulizer is completed, the residual medicament is reduced, and the problems of wasting medicament or supplying insufficient medicament can be overcome. Moreover, since the inclination angle is specially designed, the residual medicament percentage can be effectively reduced, and the overall height and size of the nebulizer and the storage chamber of the medicament storage module can be reduced while complying with the required medicament capacity (e.g. 4˜5 ml). Consequently, the nebulizer can be carried by the user more easily. In the subsequent use of the nebulizer, the problem of contaminating the medicament or causing bacteria growth will be avoided. Moreover, the nebulizer of the preset invention can be used to administer medicament to treat or alleviate pain of the patient with a respiratory disease. In addition, the nebulizer can be used to administer medicament to be inhaled into the lungs of the patient in replace of oral administration. Consequently, the absorption efficiency and action efficiency of the medicament are enhanced and the occurrence of the side effect is reduced. Moreover, the nebulizer of the present invention can be carried to immediately administer medicament and quickly generate the drug action to the patient with exacerbation of the obstructive respiratory tract. In other words, the use of the nebulizer can immediately cure or relieve the patient in the event of sudden onset.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A nebulizer, comprising:
a medicament storage module comprising:
an inclined bottom wall inclined at an inclination angle with respect to a horizontal plane;
a tubular sidewall connected with a periphery of the inclined bottom wall, wherein a storage chamber with a top entrance is defined by the inclined bottom wall and the tubular sidewall collaboratively, and a liquid medicament is accommodated within the storage chamber, wherein the tubular sidewall has an outlet, and the inclined bottom wall is extended downwardly to the outlet; and
a guiding part arranged around a junction between the inclined bottom wall and the tubular sidewall; and
a nebulizing unit disposed on the medicament storage module and aligned with the outlet, wherein the liquid medicament outputted from the outlet is nebulized into a plurality of mist droplets by the nebulizing unit.

2. The nebulizer according to claim 1, wherein the inclination angle is in a range between 3 and 30 degrees.

3. The nebulizer according to claim 1, wherein the inclination angle is in a range between 5 and 15 degrees.

4. The nebulizer according to claim 1, wherein the outlet has a first opening and a second opening, wherein the first opening is open to an inner surface of the tubular sidewall and aligned with a notch of the guiding part, and the second opening is open to an outer surface of the tubular sidewall.

5. The nebulizer according to claim 4, wherein both sides of the notch of the guiding part have beveled edges.

6. The nebulizer according to claim 1, wherein the guiding part is a C-shaped curvy structure.

7. The nebulizer according to claim 1, wherein the outlet is close to a lowest position of a bottom surface of the storage chamber.

8. The nebulizer according to claim 1, wherein the medicament storage module further comprises an upper cover, wherein the upper cover is pivotally coupled to the tubular sidewall for sheltering the top entrance of the storage chamber.

9. The nebulizer according to claim 8, wherein the tubular sidewall further comprises at least one first engaging element, and the upper cover comprises at least one second engaging element corresponding to the at least one first engaging element, wherein the at least one first engaging element and the at least one second engaging element are engaged with each other.

10. The nebulizer according to claim 1, wherein the medicament storage module further comprises a coupling part, and the coupling part is connected with an outer surface of the tubular sidewall and aligned with the outlet, wherein the coupling part comprises an extension wall and a first recess, and the nebulizing unit is disposed within the first recess.

11. The nebulizer according to claim 10, further comprising a conduit, wherein the conduit is detachably connected with the coupling part of the medicament storage module.

12. The nebulizer according to claim 1, wherein a second recess is formed in a top edge of the tubular sidewall, and a waterproof gasket is accommodated within the second recess.

13. The nebulizer according to claim 1, wherein the nebulizing unit comprises a piezoelectric element and a nozzle plate.

14. A medicament storage module, comprising:
an inclined bottom wall inclined at an inclination angle with respect to a horizontal plane;
a tubular sidewall connected with a periphery of the inclined bottom wall, wherein a storage chamber with a top entrance is defined by the inclined bottom wall and the tubular sidewall collaboratively, and a liquid medicament is accommodated within the storage chamber, wherein the tubular sidewall has an outlet, and the inclined bottom wall is extended downwardly to the outlet; and
a guiding part arranged around a junction between the inclined bottom wall and the tubular sidewall.

15. The medicament storage module according to claim 14, wherein the inclination angle is in a range between 3 and 30 degrees.

16. The medicament storage module according to claim 14, wherein the inclination angle is in a range between 5 and 15 degrees.

17. The medicament storage module according to claim 14, wherein the guiding part has a notch aligned with the outlet, and both sides of the notch have beveled edges.

18. A medicament storage module, comprising:
an inclined bottom wall inclined at an inclination angle with respect to a horizontal plane;
a tubular sidewall; and
a C-shaped curvy structure arranged around a junction between the inclined bottom wall and the tubular sidewall, wherein a storage chamber with a top entrance and an outlet is defined by the inclined bottom wall, the tubular sidewall and the C-shaped curvy structure collaboratively, and a liquid medicament is accommodated within the storage chamber, wherein the inclined bottom wall is extended downwardly to the outlet.

19. The medicament storage module according to claim 18, wherein the inclination angle is in a range between 3 and 30 degrees.

20. The medicament storage module according to claim 18, wherein the inclination angle is in a range between 5 and 15 degrees.
